SEQUENCE LISTING (12 sequences)

SEQ ID NO: 1 - DNA, 54 bp, Artificial
  Primer LES 21
gttggtcgctgcttcccaagctgccttaggtttgtaataagcttaattcttatg

SEQ ID NO: 2 - DNA, 54 bp, Artificial
  Primer LES 22
cataagaattaagcttattacaaacctaaggcagcttgggaagcagcgaccaac

SEQ ID NO: 3 - DNA, 38 bp, Artificial
  Primer Fwd rHA single FLAG
ttaggcttagattataaagatgatgacgataaataata

SEQ ID NO: 4 - DNA, 39 bp, Artificial
  Primer Rev rHA single FLAG
agcttattatttatcgtcatcatctttataatctaagcc

SEQ ID NO: 5 - DNA, 774 bp, Artificial
  Synthetic construct
agatctctgcagaagttcaattgttggaatctggtggtggtttggttcaacctggtggttctttgagattgtcttgtgctgcttctggttttactttttctaattattggatgtcttgggttagacaagctccaggtaaaggtttggaatgggtttccggtatttcaggtaatggtggttatacttattttgctgattcagttaaagatagatttactatttctagagataattctaaaaataccttatatttgcaaatgaactctttgagagcagaagatactgctgtttattactgtgcaggtggtgacggttctggttggagtttttggggtcaaggtactctagttaccgtttcttcaggtggtggtggttctggtggaggtggatcaggtggtggaggatctcaatcagttttgactcaaccaccatctgcttcaggtactccaggtcaaagagttaccatttcttgtactggttcttcttctaatattggtgcaggttacgatgttcattggtatcaacaattgccaggtactgctccaaaattgttgatttatggtaacaacaatagaccatctggtgtcccagatagattttctggttctaaatctggtacttctgcttctttggctatttctggtttaagatcagaagatgaagctgattactactgtgctgcttgggatgactctttgtctggtagagttttcggtggtggtactaaattgaccgttttgggtgacgctcacaagtccgaagtcgctcatcgat

SEQ ID NO: 6 - DNA, 91 bp, Artificial
  Primer CF 138
agcttaacctaattctaacaagcaaagatgcttttgcaagccttccttttccttttggctggttttgcagccaagatctctgcagaagaca

SEQ ID NO: 7 - DNA, 91 bp, Artificial
  Primer CF 139
agcttgtcttctgcagagatcttggctgcaaaaccagccaaaaggaaaaggaaggcttgcaaaagcatctttgcttgttagaattaggtta

SEQ ID NO: 8 - DNA, 389 bp, Artificial
  synthetic
gaattcaatcagtaaaaatcaacggttaacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgaagctttaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccgaattc

SEQ ID NO: 9 - DNA, 1328 bp, Artificial
  Synthetic construct
ccatggtcactcttaaagagaagctgttagtggcctgtcttgctgtctttacagcggtcattagattgcatggcttggcatggcctgacagcgtggtgtttgatgaagtacatttcggtgggtttgcctcgcaatacattagggggacttacttcatggatgtgcatcctcctcttgcaaagatgttgtatgctggtgtggcatcgcttggtgggttccagggtgattttgacttcgaaaatattggtgacagctttccatctacgacgccatacgtgttgatgagatttttctctgcttctttgggggctcttactgttattttgatgtacatgactttacgttattctggtgttcgtatgtgggttgctttgatgagcgctatctgctttgccgttgaaaactcgtacgtcactatttctcgttacattctgttggacgccccattgatgtttttcattgcagctgcagtctactctttcaagaaatacgaaatgtaccctgccaactcgctcaatgcttacaagtccttgcttgctactggtattgctcttggtatggcatcttcatccaaatgggttggtcttttcacggttacatgggtgggtcttttatgtatctggagactatggttcatgattggggatttgactaagtcttccaagtccatcttcaaagtagcatttgccaaattggccttcttgttgggtgtgccttttgccctttatctggtcttcttttatatccacttccaatcattaactttggacggggatggcgcaagcttcatttctaaatttattgaatcccataaaaagatgtggcatatcaataaaaatttggtcgaacctcatgtttatgaatcacaaccaacttcatggccattcttgctacgtggtataagttactggggtgaaaataacagaaacgtctatctattaggtaatgcgatcgtatggtgggctgtcaccgctttcatcggtattttcggattgattgttatcactgagctgttctcgtggcagttaggtaaaccaattttgaaggactccaaggttgttaacttccacgttcaggttattcactacttattgggttttgccgtccattatgctccatctttcttaatgcaacgtcaaatgtttttgcatcactacttacctgcttattatttcggtattcttgcccttggccacgccttggacataatagtttcttatgttttccgcagcaagagacaaatgggctacgcggtagtgatcactttccttgctgcttctgtgtatttcttcaagagcttcagtccaattatttacggtacaccatgg

SEQ ID NO: 10 - DNA, 865 bp, Artificial
  synthetic
aagctttcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgcactagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaagccttttgaaaagcaagcataaaagatcaagctt

SEQ ID NO: 11 - DNA, 1378 bp, Artificial
  Synthetic construct
agatctggtatccaaaagaagttgtttttgatgaggtacatttcgggaaatttgcatcgtattacttagaaaggtcttatttctttgacgttcatcccccttttgctaagatgatgattgccttcattggttggttatgtggctatgatggttcctttaagtttgatgagattgggtattcttatgaaactcatccagctccatatatcgcgtaccgttctttcaacgcgatattgggcacattgactgtaccaattatgttcaacactttgaaggaactgaatttcagggctattacatgtgcgtttgcatctctcttggttgcaatcgatactgcgcatgttacagaaactaggctgattttactggatgccatcttgattatttctattgctgctactatgtattgttacgttcgtttctacaagtgccaattgcgtcaaccttttacatggagttggtatatttggttacacgctactggtttgtctttatccttcgtgatttccacaaaatatgttggtgttatgacatattccgctattggttttgctgctgtggtcaacttatggcaattactggacatcaaggcgggtttgtccttgaggcagttcatgagacattttagtaaaaggctgaatggtttagttttgattccatttgtgatttacttgttttggttctgggttcatttcaccgttttgaatacttcaggtcctggcgacgcaagcttaagccattaccattcttgaagaaatggattgaaactcaaaaatctatgttcgaacataacaataaactatcatcagagcatccatttgcctctgaaccttacagttggcccggtagtttaagtggtgtttcgttctggaccaacggtgacgaaaagaagcaaatatatttcattggtaacatcattgggtggtggttccaagtcatatcattggctgtttttgttggcattatcgtggccgatttaattactagacatcgtggctattatgccctaaacaagatgaccagagaaaagctgtatggcccattgatgtttttcttcgtctcctggtgctgtcattattttccattctttttaatggcgcgtcaaaagtttttgcatcattacttaccagctcatttaatcgcgtgcttattctcaggagcactatgggaagtaattttcagtgattgcaaatcattggatttggagaaagacgaggatatttcaggtgcatcatatgaacggaaccctaaggtctacgttaaaccctataccgtcttcttggtgtgtgtctcctgtgctgttgcgtggttttttgtatacttttcaccactagtgtatggagatgtcagcttgtcaccatcggaagttgtttctaga

SEQ ID NO: 12 - protein, 244 aa, Artificial
  Synthetic construct
EVQLLESGGGLVQPGGSLRLSCAASGFTFSNYWMSWVRQAPGKGLEWVSGISGNGGYTYFADSVKDRFTISRDNSKNTLYLQMNSLRAEDTAVYYCAGGDGSGWSFWGQGTLVTVSSGGGGSGGGGSGGGGSQSVLTQPPSASGTPGQRVTISCTGSSSNIGAGYDVHWYQQLPGTAPKLLIYGNNNRPAGVPDRFSGSKSGTSASLAISGLRSEDEADYYCAAWDDSLSGRVFGGGTKLTVLG

(19) United States
(12) Patent Application Publication
(10) Pub. No.: US 2012/0059155 A1
(43) Pub. Date: Mar. 8, 2012

(54) Method of Controlling O-Linked Glycosylation of Antibodies

(75) Inventors: Leslie Robert Evans, Nottingham (GB); Miranda Hughes, Notts (GB); Joanna Hay, Devon (GB); Darrell Sleep, Nottingham (GB); David John Tooth, Nottingham (GB); Neil Dodsworth, Nottingham (GB); Malcolm John Saxton, Nottingham (GB); Joanne Patricia Waters, Nottingham (GB); Steven Athwal, Nottingham (GB)

(73) Assignee: NOVOZYMES BIOPHARMA DK A/S, Bagsvaerd (DK)

(21) Appl. No.: 13/319,019

(22) PCT Filed: May 7, 2010
(86) PCT No.: PCT/EP10/56266

§ 371 Date: Nov. 4, 2011

(30) Foreign Application Priority Data
May 7, 2009 (EP) 09159641.1

(51) Int. Cl. C07K 16/00; C12P 21/00

(52) U.S. Cl. 530/387.3; 435/69.6; 530/387.1

(57) ABSTRACT

A method for producing antibodies in fungal host cells is provided where the produced antibodies has a low degree of glycosylation.

### REFERENCE TO A SEQUENCE LISTING

This application contains a Sequence Listing in computer readable form. The computer readable form is incorporated herein by reference.

### FIELD OF THE INVENTION

The present invention relates to a method for controlling O-linked glycosylation of polypeptides produced recombinantly in fungi. The invention relates in particular to the production of antibodies having a low degree of glycosylation.

### BACKGROUND OF THE INVENTION

Recombinant proteins are now widely used in many fields including; commercial and industrial enzymes, diagnostics, analytics, downstream purification, formulation, as vaccines and as therapeutic agents.

Traditionally, and prior to the advent of recombinant DNA technology, mammalian proteins have been obtained from mammalian origins. However, it follows that proteins derived from mammalian origins inevitably risk being infected or contaminated with potentially deleterious, or adventitious viruses, prions or other harmful agents. These methods have proved to be both expensive and in many cases non reproducible. Consequently, mammalian cell culture techniques have been used as a source for mammalian proteins, but culturing mammalian cells is costly, technically difficult and the yields are often modest.

For these, as well as other reasons, such as easy scale up and rapid growth rates, microbial expression systems have the potential to be viable alternatives in the production of biopharmaceutical and biotherapeutic proteins including antibodies antibody fragments and antibody fusions amongst other commercially relevant proteins. The production of antibodies and antibody fragments is discussed in Joosten et al., Microbial Cell Factories (2003), 2, 1-15 and Gasser and Mattanovich, Biotechnol. Lett. (2007) 29, 201-212.

However, the phenomenon of O-linked glycosylation as a post-translational modification of recombinant proteins for potential pharmaceutical manufacture and therapeutics, is a concern and has implications for, among other things, the heterogeneity, stability, immunogenic effect (both antigenic and immunosuppressive), structure, function, activity, secretion, therapeutic efficacy, lymphoprolific effects, and aggregation of the product. The structure of O-linked sugar chains in fungal expressed proteins are known to present themselves differently than that seen with mammalian expression systems. Similarly, heterologous proteins which would not normally be O-linked glycosylated may be when expressed in fungi. Hence, for the production of pharmaceutical grade proteins, e.g. therapeutic monoclonal antibodies (mAbs), it would be desirable to reduce the amount of glycosylation of mammalian proteins produced in fungi.

It is generally known that protein glycosylation is widespread amongst both prokaryotes and eukaryotes. This modification has been linked to cell wall integrity, cellular differentiation, virulence, secretion and development. Glycosyl residues can be linked to proteins via asparagine (N-glycosylation) or via hydroxylated amino acids, primarilarly serine and threonine, but more rarely, tyrosine, hydroxyproline and hydroxylysine (O-glycosylation). Various monosaccharides can be O-linked to proteins, including galactose (Gal), glucose (Glc), N-acetylgalactosamine (GalNAc) and mannose (Man). These then can be extended further by additional sugars, attached via O-glycosidic bonds, mediated by mannosyl transferases, or other specific sugar transferases. (ref: Lussier, M. et al (1999) Biochim. Biophys. Acta 1426, 323-334). There has therefore been considerable interest in providing methods for reducing the mannose type glycosylation of mammalian proteins, such as antibodies, produced in fungi.

### SUMMARY OF THE INVENTION

The invention relates to a method for preparing a polypeptide comprising an antibody sequence or a fragment thereof, said polypeptide having a low degree of O-linked glycosylation, comprising the steps of:
a. providing a nucleic acid sequence encoding a polypeptide comprising an antibody sequence or a fragment thereof;
b. modifying the nucleic acid sequence so that at least one amino acid residue selected among S, T and Y and subject to O-linked glycosylation is substituted or deleted;
c. introducing the modified nucleic acid sequence in a suitable host cell so that the modified nucleic acid sequence is capable of being expressed in the host cell;
d. growing the host cell under conditions leading to expression of the polypeptide encoded by the modified nucleic acid sequence, and
e. recovering the polypeptide.

The invention further relates to polypeptides obtainable according to a method of the invention. Compositions comprising polypeptides prepared according to a method according to the invention are further disclosed.

### BRIEF DESCRIPTION OF DRAWINGS

FIG. 1 shows Plasmid map of pDB3017

FIG. 2 shows LC-MSMS spectra of 703.63+ion (180 LLIYGNNNRPSGVPDR195) from tryptic digest of DXY1 expressed the anti-FITC ScFv rHA fusion.

FIG. 3 shows plasmid map of pDB2777

FIG. 4 shows plasmid map of pAYE587

FIG. 5 shows plasmid map of pDB2779

FIG. 6 shows plasmid map of pDB3070

FIG. 7 shows plasmid map of pDB3088

FIG. 8 shows plasmid map of pDB3979

FIG. 9 LC-MS spectra of 632.34³⁺ ion (₀EVQLLESGGGLVQPGGSLR₁₉)from tryptic digest of DYP4 expressed the anti-FITC scFv S190A mutant rHA fusion. Tryptic peptides were ConA enriched before BEMAd1 treatment and subsequent LCMSMS analysis. BEMAd 1 one treatment of a peptide containing a single site of O-linked mannosylation would result in a mass shift of one Da (632.34³⁺). The above spectra confirms a one Da loss from the peptide.

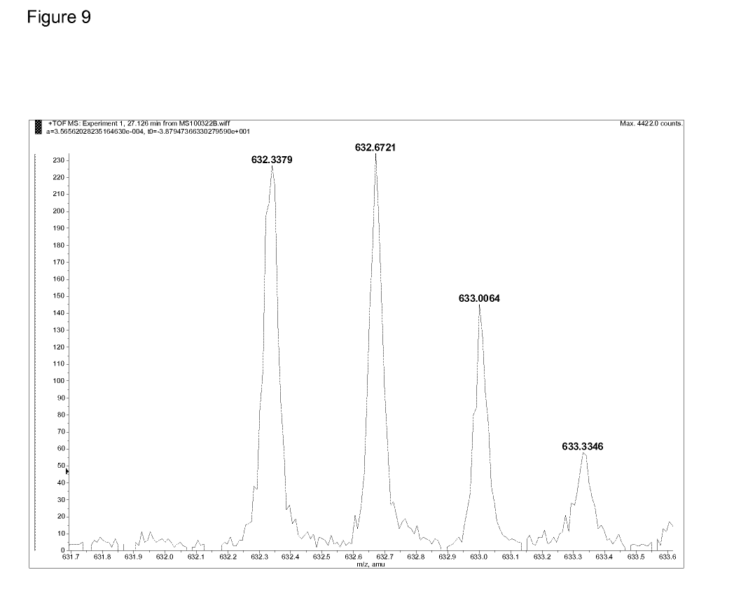

FIG. 10 LC-MSMS spectra of 632.34³⁺ion (₀EVQLLESGGGLVQPGGSLR₁₉)from tryptic digest of DYP4 expressed the anti-FITC scFv S190A mutant rHA fusion. Tryptic peptides were ConA enriched before BEMAd1 treatment and subsequent LCMSMS analysis. All major ions correspond to expected b and y type ions for the s7 dehydro peptide. B ions 10,11 and 12 with the dehydro modification present are diagnostic for the presence of a dehydro serine and position 7 and therefore pre-BEMAd1 treatment an o-linked mannose moiety. Confirming S7 as a site of o-linked mannosylation.

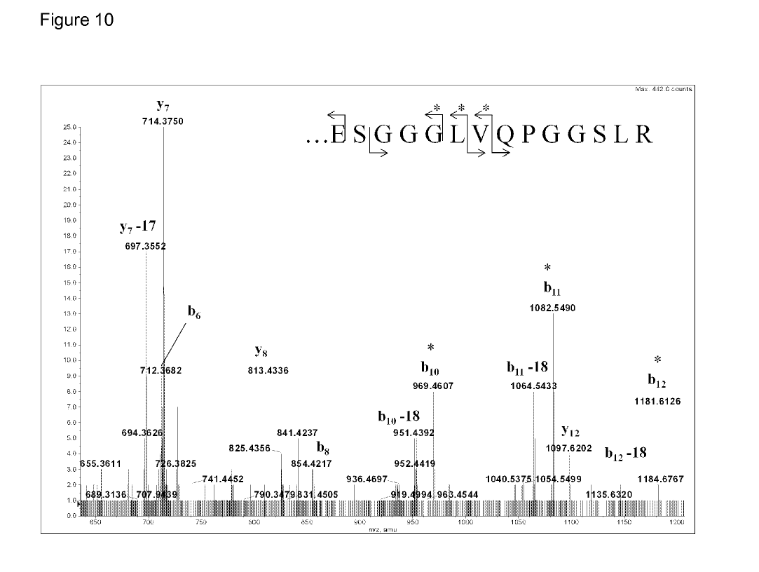

FIG. 11 LC-MSMS spectra of 772.382⁺ ion (₂₀₁SGTSASLAISGLR₂₁₃)from tryptic digest of DYP4 expressed the anti-FITC scFv S190A mutant rHA fusion. Tryptic peptides were ConA enriched before LCMSMS analysis. The majority of ions seen in the spectra corresponded to the un-mannosylated species (where the labile mannose had fallen off). However the presence of y8, y9 and y10 with mannose still present confirms that at least on mannose is present at serine 206 or serine 210 the absence of any mannose still present on y7, y6, y5 or y5 despite the high abundance of the fragment ions with no mannose present is highly indicative of the presence of o-linked mannose of serine 206.

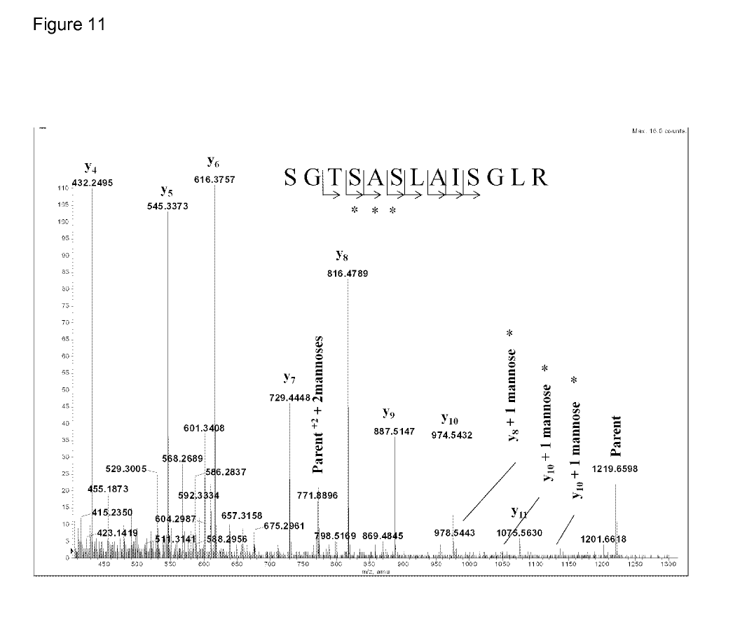

### DETAILED DESCRIPTION OF THE INVENTION

The term “antibody” or “antibody molecule” as used herein is thus intended to include whole antibodies (e.g. Immunoglobulin G (IgG), Immunoglobulin A (IgA), Immunoglobulin E (IgE), Immunoglobulin M (IgM), or Immunoglobulin D (IgD)), (mAbs), polyclonal antibodies, and chimeric antibodies. The Immunoglobulin (Ig) classes can be further divided into subclasses on the basis of small differences in the amino acid sequences in the constant region of the heavy chains. Igs within a subclass can have similar heavy chain constant region amino acid sequences, wherein differences are detected by serological means. For example, the IgG subclasses comprise IgG1, IgG2, IgG3, and IgG4, wherein the heavy chain is classified as being a gamma 1 heavy chain, a gamma 2 heavy chain, and so on, due to the amino acid differences. The light chain can also be of the kappa or lambda type. In another example, the IgA subclasses comprise IgA1 and IgA2, wherein the heavy chain is classified as being an alpha 1 heavy chain or an alpha 2 heavy chain due to the amino acid differences. Antibodies in this context are also intended to include those devoid of light chains, such as those found in camel, llama and other members of the camelidae family, and sometimes referred to as heavy chain antibodies (HcAb). Similarly, Immunoglobulin isotype novel (or new) antigen receptors (IgNAR's), which are naturally found in cartilaginous marine animals, for example wobbegong sharks and nurse sharks, and other members of the Chondrichthyes class (cartilaginous fishes).

Antibody fragments which comprise an antigen binding domain are also included. The term “antibody fragment” as used herein is intended to include any appropriate antibody fragment that displays antigen binding function. Several such antibody fragments have been described in the art and are known as Fab, F(ab′)2, Fab3, scFv, Fv, dsFv, ds-scFv, Fd, dAbs, TandAbs, flexibodies dimers, minibodies, diabodies, tribodies, tetrabodies, vH domain, vL domain, v_{H}H domain, Nanobodies, IgNAR variable single domain (v-NAR domain), fragments thereof, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

The antibodies, antibody fragments, or antibody fusions, are according to the invention produced recombinantly in a suitable host cell. The antibodies, antibody fragments, or antibody fusions may be produced recombinantly in their final form or they may be produced in a form that can be converted into the final desired antibody, antibody fragment, or antibody fusion by one or more subsequent steps. For example, an antibody fragment according to the invention may be produced recombinantly as a whole antibody in a suitable host cell, and then converted into the desired antibody fragment using conventional techniques e.g. cleavage with a protease. Preferably the antibody, antibody fragment, or antibody fusion comprises an antibody light chain variable region (vL) and/or an antibody heavy chain variable region (vH), which generally comprise the antigen binding site. In certain embodiments, the antibody, antibody fragment, or antibody fusion comprises all or a portion of a heavy chain constant region, such as an IgG1, IgG2, IgG3, IgG4, IgA1, IgA2, IgE, IgM or IgD constant region. Preferably, the heavy chain constant region is an IgGI heavy chain constant region. Furthermore, the antibody, antibody fragment, or antibody fusion can comprise all or a portion of a kappa light chain constant region or a lambda light chain constant region. Preferably, the light chain constant region is a lambda light chain constant region. All or part of such constant regions may be produced naturally or may be wholly or partially synthetic. Appropriate sequences for such constant regions are well known and documented in the art.

The term “fragment” as used herein refers to fragments of biological relevance, e.g. fragments which can contribute to or enable antigen binding, e.g. from part or all of the antigen binding site, or can contribute to the inhibition or reduction in function of the antigen or can contribute to the prevention of the antigen interacting with its natural ligands. Preferred fragments thus comprise a heavy chain variable region (vH domain) and/or a light chain variable region (vL domain) of the antibodies of the invention. Other preferred fragments comprise one or more of the heavy chain complementarity determining regions (CDRs) of the antibodies of the invention (or of the vH domains of the invention), or one or more of the light chain CDRs of the antibodies of the invention (or of the vL domains of the invention). When used in the context of a nucleic acid molecule, the term “fragment” includes a nucleic acid molecule encoding a fragment as described herein.

The term “antibody sequence” is intended to mean the polypeptide sequence of an antibody comprising both CDR and framework sequences.

The phrase “immunoglobulin single variable domain” refers to an antibody variable region (vH, v_{H}H, vL) that specifically binds an antigen or epitope independently of other v regions or domains; however, as the term is used herein, an immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, preferably human, but also includes rodent (for example, as disclosed in WO00/29004, the contents of which are incorporated herein by reference in their entirety), camelid v_{H}H dAbs or cartilaginous marine animal-derived immunoglobulin-like molecules, for example as disclosed in WO/2009/026638 and WO2005/118629, the contents of which are incorporated herein by reference in their entirety. Camelid dAbs are immunoglobulin single variable domain polypeptides which are derived from species including camel, llama, alpaca, dromedary, and guanaco, and comprise heavy chain antibodies naturally devoid of light chain: v_{H}H- v_{H}H molecules are about ten times smaller than IgG molecules.

The term “domain” in the present invention, with regards to the immunogolobulins is a folded protein structure which retains its tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

The term “Immunoglobulin” refers to a family of polypeptides which retain the immunoglobulin fold characteristic of antibody molecules, which contains two [beta] sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules. Preferably, the present invention relates to antibodies.

A “universal framework” or “framework” is a single antibody framework sequence corresponding to the regions of an antibody conserved in sequence as defined by Kabat. The Kabat system/scheme (a well known and widely used guide) is used to identify framework regions and CDRs of the invention—see Sequences of Proteins of Immunological Interest, E. Kabat et al., U.S. Department of Health and Human Services, (1987) and (1991). Identifying Kabat frame-work sequence is well known and thus is a routine protocol; see e.g., U.S. Pat. No. 5,840,299; U.S. Pat. App. Pub. No. 20050261480. Kabat et al. list many amino acid sequences for antibodies for each subclass, and list the most commonly occurring amino acid for each residue position in that subclass. Kabat et al. use a method for assigning a residue number to each amino acid in a listed sequence, and this method for assigning residue numbers has become standard in the field. Kabat et al.'s scheme is extendible to other antibodies, antibody fragments and antibody fusions not included in the compendium by aligning the antibody, antibody fragment, or antibody fusion, in question with one of the consensus sequences in Kabat et al. The use of the Kabat et al. numbering system readily identifies amino acids at equivalent positions in different antibodies, antibody fragments and antibody fusions. For example, an amino acid at the L50 position of a human antibody occupies the equivalent position to an amino acid position L50 of a mouse antibody.

In another example, the amino acid residues of a v_{H}H domain from Camelids (ref: Riechmann and Muyldermans (2000), J. Immunol. Meth. 240 185-195) can be numbered according to the general numbering for vH domains given by Kabat et al. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-1 13. In this, and other respects, it should be noted that it is well known in the art for vH domains and for vHH domains, that the total number of amino acid residues in each of the CDR's may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering. This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR' s, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa. This concept is further outlined in WO2009004066

Natural antibodies are polypeptides produced in vivo by an organism and which can be secreted into the plasma in response to exposure to an allergen or antigen, which polypeptides have the ability to bind specifically to said antigen. Natural antibodies include but are not limited to Igs, such as IgMs, IgDs, IgGs, IAgs and IgEs.

Artificial antibodies are antibodies wherein the CDRs occur together with framework sequences with which they are not naturally connected.

Artificial antibodies comprises modifications, fragments, variants, mutants, homologs and analogs or combinations of modifications, fragments, variants, mutants, homologs and analogs of natural antibodies which retain the ability to bind specifically to an antigen. Artificial antibodies includes but are not limited to fragments and variants known in the art as Fab fragments, F(ab)2 fragments and scFv fragments. All have domains with Ig, or Ig-like folds, which consist of a beta sandwich of seven or more strands in two sheets with a Greek-key topology.

Fusions of antibodies are according to the invention intended to mean a polypeptide comprising one or more antibodies, or antibody fragment sequences and one or more sequences not derived from antibodies, which fusion is capable of binding to an antigen. Typically the one or more sequences not derived from antibodies are derived from a plasma protein such as albumins, transferrins (US2008/0220002), lactoferrins or melanotransferrins. The sequences not derived from an antibody comprise preferably at least 10 amino acids, at least 20 amino acids, 30 amino acids, more preferred at least 50 amino acids, even more preferred at least 75 amino acids and more preferred at least 100 amino acids.

The antibody fusion may be a N-terminal fusion or a C-terminal fusion or a N- and C-terminal fusion understood as a fusion where the antibody sequence is fused N-, C-or N- and C-terminally to the non antibody sequence. The antibody sequence may also be inserted internally into the non antibody sequence such as in a loop or a structure known to be located on the surface of the molecule comprising said non antibody sequence. The fusion may further comprise linker sequences between the antibody and non-antibody sequences. This concept is further outlined in WO 01/79442.

In one preferred embodiment the one or more antibody sequences of an antibody fusion is an antibody fragment, such as a Fab fragment, F(ab)2 fragment and scFv fragment. In another preferred embodiment the one or more sequences not derived from antibodies is an albumin or a fragment thereof. In a particular preferred embodiment the antibody fusions comprise a scFv sequence and human serum albumin or a fragment thereof.

Preferred framework sequences according to the invention are sequences having at least 60% sequence, preferred at least 70% identity, more preferred at least 80% identity, more preferred at least 85% identity, more preferred at least 90% identity, even more preferred at least 95% identity most preferred at least 97% identity to any one of the framework sequences from mammalian, e.g. mouse, rat, rabbit, sheep, bovine, ovine, equine, avian, primate, human; IgG, IgA, IgM, IgD or IgE or any consecutive 25 amino acid sequence fragment thereof.

Examples of framework sequences include the framework sequences derived from IgG, IgA, IgM, IgD or IgE antibodies and any fragments thereof derived from mammals in particular from mouse, rats, rabbits, guinea pigs, and primates, in particular primates such as homo sapiens. Similarly, framework sequences derived from Ig superfamilies found in members of the camelidae family, e.g. llamas, and members of the Chondrichthyes class, e.g. sharks.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http:/emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment).

As described herein the term “antigen” is understood in the usual way as a molecule that is bound by a binding domain according to the present invention. Typically, antigens are bound by antibody (and fragments therein) ligands.

As used in the art, the term “CDR” refers to a complementarity determining region within antibody variable sequences. There are three CDRs in each of the variable regions of the heavy chain and the light chain, which are designated CDR1, CDR2 and CDR3 for each of the variable regions. Because CDRs represent regions of increased variability (relative to the regions of similar sequences), the exact boundaries of these CDRs can be defined differently according to different systems. The widely used system described by Kabat (Kabat et al.). Sequences of Proteins of Immunological Interest (National Institutes of Health, Bethesda, Md. (1987) and (1991)) provide a residue numbering system applicable to any variable region of an antibody, and provide residue boundaries defining the three CDRs. These CDRs may be referred to as “Kabat CDRs”. Chothia et al. (Nature (1989) 342:877-883; Chothia and Lesk, (1987)/. Mol. Biol. 196:901-917) found that certain sub-portions within Kabat CDRs adopt nearly identical peptide backbone conformations, despite having great diversity at the level of amino acid sequence. These subportions were designated as L1, L2 and L3 or H1, H2 and H3 where the “L” and the “H” designate the light chain and the heavy chains regions. These regions may be referred to as Chothia CDRs, which have boundaries that overlap with Kabat CDRs. The term “framework,” “framework region,” or “framework sequence” refers to the remaining sequences of a variable region minus the CDRs. Because the exact definition of a CDR sequence can be determined by different systems, the meaning of a framework sequence is subject to correspondingly different interpretations. In one embodiment, the positioning of the six CDRs (CDR1, 2, and 3 of light chain and CDR1, 2, and 3 of heavy chain) within the framework region effectively divides the framework region of each chain into four subregions, designated FR1, FR2, FR3, and FR4. CDR1 is positioned between FR1 and FR2; CDR2 between FR2 and FR3; and CDR3 between FR3 and FR4. Without specifying the particular subregions as FR1, FR2, FR3, or FR4, a framework region, as referred by others, represents the combined subregions FR1, FR2, FR3, and FR4, within the variable region of a single, naturally occurring immunoglobulin chain. In an alternative embodiment, a framework region (FR) of the invention comprises or consists of (represents) any portion of the entire framework sequence, including a sequence consisting of one of the four subregions. In an alternative embodiment, a framework region (FR) of the invention comprises or consists of amino acids derived from a Kabat framework region (KF) domain, wherein the amino acid sequences are derived from germline immunoglobulin sequences.

In this application the Kabat numbering system will be used for identification of amino acid sequences within antibody sequences according to the method disclosed in Martin, A. C. R. Accessing the Kabat Antibody Sequence by Computer. PROTEINS: Structure, Function and Genetics, 25 (1996), 130-133.

In an antibody several loops have been identified as comprising CDR sequences and being involved in antigen binding. The Kabat numbering assigns the following numbering to CDR's:

| Loop | Number |
|---|---|
| L1 | L24-L34 |
| L2 | L50-L56 |
| L3 | L89-L97 |
| H1 | H31-H35B |
| H2 | H50-H65 |
| H3 | H95-H102 |

According to the invention at least one amino acid residue subjected to O-linked glycosylation is substituted with an amino acid that does not allow O-linked glycosylation, or is deleted. O-linked glycosylation is defined as the attachment of a sugar group, usually a mannose residue, to an amino acid in a polypeptide sequence where the sugar group is attached via an O atom of the amino acids side chain. For example, this type of glycosylation is seen in yeast and filamentous fungi. O-linked glycosylation occurs on one of the amino acid residues S, T or Y, however, not all S, T or Y residues in a polypeptide produced in a fungal host cell will be glycosylated and a reliable prediction of which S, T or Y residues in a given polypeptide sequence is not yet possible.

A residue subjected to glycosylation may be identified using different technologies known in the art. In one method the polypeptide is digested with an endopeptidase generating peptides of a suitably small size. The peptides containing attached sugar moieties are recovered from the digest e.g. using a matrix having attached a ligand with affinity for sugar moieties such as concanavalin A (Con A), and the recovered peptides are sequenced for identifying the amino acids having sugar groups attached. Other techniques known to the skilled person for identifying residues prone to O-linker glycosylation may also be applied to the invention.

Once a suitable residue subjected to O-linked glycosylation has been identified it is according to the invention deleted or substituted to an amino acid not subjected to O-linked glycosylation i.e. to any amino acid selected among A, C, D, E, F, G, H, I, K, L, M, N, P, Q, R, V or W. It is preferred to substitute the S, T, or Y residue to a different residue having a similar size, and further it is preferred not to substitute the amino acids to a charged residue. Thus preferably S or T is substituted with A, C, G or V, and Y is preferably substituted to F, I, L, M, N, Q or W.

The deletion or substitution of one or more amino acid residues subjected to O-linked glycosylation is conveniently done using well known techniques for modifying nucleic acid sequences, such as site directed mutagenesis. A multitude of techniques for modifying nucleic acids are known in the art and the skilled person will know how to apply these techniques to the present invention.

The one or more amino acid residue subjected to O-linked glycosylation can in principle be located at any position in the antibodies. It is preferred that the one or more amino residues subjected to O-linked glycosylation are located in the framework region of the antibody. As examples of suitable amino acid residues subjected to O-linked glycosylation which residue suitably may be substituted or deleted according to the invention can be mentioned the residue having the number L56 using the Kabat numbering scheme. The residue having the number L56 is within one of the CDRs' meaning that this particular residue is located in a highly variable region of the antibody. Therefore, there will exist antibodies having a different amino acid residue than S, T or Y in position L56. The skilled person will appreciate that this embodiment only applies to antibodies having a S, T or Y residue in position L56.

Other preferred examples of suitable amino acids residues subject to O-linked glycosylation are Serine 7 and Serine 206 in SEQ ID NO: 12, Serine 7 and Serine 206 in the Anti MUC1 scFv ((ref: British J. of Cancer (1997) 76 (5) 614-621), Serine 7 and Serine 191 in the Anti 2,4-D scFv (ref: Vet. Med.—Czech, 48, 2003 (9): 237-247), and Threonine 72 in the Anti IL-1R1 dAb (ref: Patent: WO 2007/063311), respectively, as well as serines in other antibodies in positions corresponding to positions 7 or 206 in Anti MUC1 scFv ((ref: British J. of Cancer (1997) 76 (5) 614-621), positions 7 or 191 in the Anti 2,4-D scFv (ref: Vet. Med. —Czech, 48, 2003 (9): 237-247), and threonine in a position corresponding to position 72 in the Anti IL-1 R1 dAb (ref: Patent: WO 2007/063311).

Particular preferred amino acid residues subjected to O-linked glycosylation, which residues suitable may be substituted or deleted according to the invention include the amino acid residue in position L56 according to Kabat numbering, or positions corresponding to positions 7, 72, 191 or 206 in SEQ ID NO: 12.

A nucleic acid construct in relation to antibodies is according to the invention intended to be understood as a nucleic acid sequence encoding an antibody, antibody fragment, or antibody fusion in functional relation with control sequences necessary for transcription and translation of the sequence encoding said antibody. The expression “in functional relation with control sequences necessary for transcription and translation of the sequence encoding said antibody” is intended to mean that the sequence encoding the antibody, antibody fragment, or antibody fusion is placed in a suitable frame, distance and orientation with respect to control sequences such as promoters, ribosome binding sites, terminators, polyadenylation sites, enhancer sequences regulator sites etc. Teachings of nucleic acid constructs for expressing a polypeptide in a host cell is abundant in the prior art and the skilled person will appreciate how to apply such teaching to the present invention.

The nucleic acid construct encoding an antibody, antibody fragment, or antibody fusion in functional relation with control sequences necessary for transcription and translation in the selected host is introduced into the selected fungal host cell. Several techniques for introducing nucleic acids into a host cell exist, and the skilled person will appreciate that the present invention is not limited to any particular such technique but any such technique may in principle be used as long as the technique is capable of introducing the nucleic acids in the host cell. An example of such a technique could include a modified lithium acetate method (Sigma yeast transformation kit, YEAST-1, protocol 2; Ito et al, (1983) J. Bacteriol., 153, 16; Elble, (1992) Biotechniques, 13, 18).

The expression “capable of directing the expression of an antibody, antibody fragment, or antibody fusion into the host cell” is intended to mean that the sequence encoding an antibody, antibody fragment, or antibody fusion is provided with the necessary promoter, terminator, ribosome binding site, enhancer, polyadenylation sequences necessary for expressing the antibody in the selected host cell. It is within the skills of the average practitioner to select suitable regulatory sequences for a particular host cell.

When a nucleic acid sequence encoding the modified antibody, antibody fragment, or antibody fusion according to the invention has been provided it is inserted into a suitable expression construct as known in the art. Such an expression construct will comprise regulatory sequences in operational relation to the sequence encoding the modified antibody, antibody fragment, or antibody fusion. Teachings concerning expression of a nucleic acid sequence in a fungal host cell are available in the prior art and the skilled person will know how to apply these teachings to the present invention.

Once the construct has been prepared it is inserted into a suitable fungal host cell.

According to the invention the fungal host cell may be any fungal cell capable of expressing the antibody, antibody fragment, or antibody fusion of the invention. The fungal cell may be a filamentous fungus or an yeast. As examples of filamentous fungi can be mentioned Aspergillus sp., such as A. nidulans, A. niger, A. Awamori, and A. oryzae; Trichoderma sp., such as T. reeseii, T. Longibrachiatum and T. virdee; Penicillum sp. such as P. notatum and P. chrysogenum; Fusidium sp., Fusarium sp., Scizophyllum sp., Mucor sp and Rhizopus sp. Preferred filamentous fungi host cells include A. niger, A. oryzae, A nidulans and T reeseii

As examples of yeast host cells can be mentioned Saccharomyces sp. such as S. cerevisiae and S. ovarum, Zygosaccharomyces sp., Schizosaccharomyces sp. such as S. pombe, Klyveromyces sp. such as K. lactis, Candida sp. such as C. albicans, Pichia sp, such as P. pastons. and Hansenula sp. Preferred yeast host cells include S.cerevisiae, K. lactis and P. pastoris.

The host cells may be wild type strains, meaning that they have a genetic configuration as could be isolated from nature or they may be genetic modified strains. Preferably the host strains have been modified genetically altered to make them more suited for production of heterologous proteins. Examples of such modifications include reducing the amount of proteases expressed by the host cell, modifications that increase the capacity of the host cell to produce heterologous proteins, such as an increase of foldases, chaperones etc. Teaching in the art concerning the production of heterologous proteins in fungal cell may also be applied to the present invention. A particular preferred fungal host cell is the yeast S. cerevisiae. Teachings concerning the yeast host cells disclosed in the international patent application published as WO 2009/019314, included in its entirety by reference, also apply to the present invention.

When a host cell comprising the nucleic acid construct capable of directing the expression of an antibody, antibody fragment, or antibody fusion in the selected host cell has been provided the host cell is grown under conditions allowing the expression of the antibody, antibody fragment, or antibody fusion. The growth conditions should be selected to provide sufficient nutrients to the host cell for growth and production of the desired antibody, antibody fragment, or antibody fusion and in case that the promoter directing transcription of the antibody is inducible, i.e. the activity of the promoter depends on the presence or absence of particular compounds or physical conditions, the growth conditions should be adapted to induce expression of the antibody, antibody fragment, or antibody fusion. It is within the skills of the average practitioner to select suitable growth conditions depending on the selected host cell and the nucleic acid constructs. The host cell comprising the nucleic acid construct is grown for a certain time until the antibody, antibody fragment, or antibody fusion has been produced in a satisfactory amount whereafter the antibody, antibody fragment, or antibody fusion is recovered using well known purification techniques. It is within the skills of the average practitioner to select suitable purification methodologies, for a specific expressed protein.

Unless defined otherwise, all technical and scientific terms used herein have the same meaning as commonly understood by one of ordinary skill in the art (e.g., in cell culture, molecular genetics, nucleic acid chemistry, fermentation, protein biochemistry and bioinformatics)

### EXAMPLES

### Example 1

### Preparation of FITC-rHA Fusion

Construction of scFv Albumin Fusion Expression Plasmid

FITC (Lantto & Ohlin, 2002, J. Biol. Chem. 277: 45108-45114) is a scFv molecule that had been expressed in P. pastoris and Escherichia coli that specifically binds to fluorescein isothiocyanate. It had been derived from a synthetic scFv library, constructed by shuffling human CDR sequences in a vHvL framework. Codon optimised DNA sequences that encoded for this protein was designed in conjunction with GeneArt GmbH.

The plasmid pDB2540 as described in US 2006/0241027 was used for site directed mutagenesis to introduce the favourable cloning site Bsu36I in the 3′ region of the recombinant human albumin (rHA) DNA sequence. Also an extra mutation was achieved that destroyed the extra HindIII site that was normally found next to the TAATAA stop codons. The mutagenic primers used along with the Stratagene Quickchange™ Site directed mutagenesis kit were LES21 & LES22;

LES21
(SEQ ID NO: 1)
5′-gttggtcgctgcttcccaagctgccttaggtttgtaataagcttaat
tcttatg-3′
LES22
(SEQ ID NO: 2)
5′-cataagaattaagcttattacaaacctaaggcagcttgggaagcagc
gaccaac-3′

This produced the plasmid pDB2836. The plasmid pDB2836 was then digested with the restriction endonuclease enzymes XbaI and Bsu36I and the 0.467 kb was liberated along with a Bsu36I NdeI 1.437 kb fragment from pDB2575 (as previously described in US 2006/0241027) which were then both ligated into the vector plasmid pDB2541 (as previously described in US 2006/0241027) that had similarly been digested with the restriction endonuclease enzymes XbaI and NdeI to produce a 3.353 kb vector. This ligation produced the plasmid pDB2839. Plasmid pDB2839 was digested with the restriction endonuclease enzymes SphI and NdeI to produce a 5.671 kb vector into which was ligated a 0.867 kb insert that had been digested from pDB2540 (as described above) with the restriction endonuclease enzymes SphI and NdeI also. This ligation produced the plasmid pDB2843. The oligo linkers Fwd rHA single FLAG® and Rev rHA single FLAG® were annealed and ligated into the 6.179 kb pDB2843 vector that had been digested with the restriction endonuclease enzymes HindIII partial (at position 3005) and Bsu36I. This ligation created the plasmid pDB2975.

Fwd rHA single FLAG ®
(SEQ ID NO: 3)
5′-TTAGGCTTAGATTATAAAGATGATGACGATAAATAATA-3′
Rev rHA single FLAG ®
(SEQ ID NO: 4)
5′-AGCTTATTATTTATCGTCATCATCTTTATAATCTAAGCC-3′

Overlapping oligonucleotide primers were used to create a synthetic DNA encoding the 3′ of the invertase leader sequence operationally linked to the FITC (vHvL) which was codon optimised for expression in S. cerevisiae which was then operationally linked to the 5′ of the rHA.

SEQ ID No. 5 is a DNA sequence based on the 3′ of the invertase leader sequence operationally linked to the FITC (vHvL) which is codon optimised for expression in S. cerevisiae which is then operationally linked to the 5′ of the rHA. The sequence is flanked by BglII and ClaI restriction endonuclease sites to facilitate cloning. SEQ ID No. 5 comprises a BglII restriction endonuclease enzyme cloning site to the 3′ invertase leader (signal) protein encoding sequence (nucleotides 1-11); the FITC (vHvL) protein encoding sequence which is codon optimised for expression in S. cerevisiae (nucleotides 12-743); the 5′ rHA protein encoding sequence up to an ClaI restriction endonuclease enzyme cloning site (nucleotides 744-770).

The synthetic DNA encoding the 3′ of the invertase leader sequence operationally linked to the FITC (vHvL) which was codon optimised for expression in S. cerevisiae which was then operationally linked to the 5′ of the rHA was digested with the restriction endonuclease enzymes BglII and ClaI to produce a 0.796 kb fragment; pDB2975 was digested with the restriction endonuclease enzymes ClaI and SphI to produce a 1.950 kb fragment; pDB2923 was digested with the restriction endonuclease enzymes BglII and SphI to produce the 4.214 kb vector; all three were used in a three way ligation to produce the sub-cloning plasmid pDB3006. The vector pDB2923 was created as follows; pDB2243 (as previously described in WO 00/44772) was digested with the restriction endonuclease enzymes Bsu36I and NdeI to produce a 1.088 kb fragment, pDB2836 (as described previously) was digested with the restriction endonuclease enzymes Bsu36I and XbaI to produce a 0.467 kb fragment which were both ligated into the vector pDB2541 (as described previously) that has been digested with the restriction endonuclease enzymes NdeI and XbaI to produce the 3.353 kb vector. This three way ligation produced the plasmid pDB2840. The plasmid pDB2840 was digested with the restriction endonuclease enzymes SphI and NdeI to produce a 5.326 kb vector, into which was ligated a 0.885 kb SphIlNdeI insert from pDB2540 (as described previously). This ligation produced the plasmid pDB2844. The oligo linkers CF138 and CF139 were annealed and ligated into the 6.193 kb pDB2844 vector that had been digested with the restriction endonuclease enzyme HindIII partial and treated with calf alkaline phosphatase. This ligation created the plasmid pDB2923 which was utilised above.

CF138
(SEQ ID NO: 6)
5′-AGCTTAACCTAATTCTAACAAGCAAAGATGCTTTTGCAAGCCTTCC
TTTTCCTTTTGGCTGGTTTTGCAGCCAAGATCTCTGCAGAAGACA-3′
CF139
(SEQ ID NO: 7)
5′-AGCTTGTCTTCTGCAGAGATCTTGGCTGCAAAACCAGCCAAAAGGA
AAAGGAAGGCTTGCAAAAGCATCTTTGCTTGTTAGAATTAGGTTA-3′

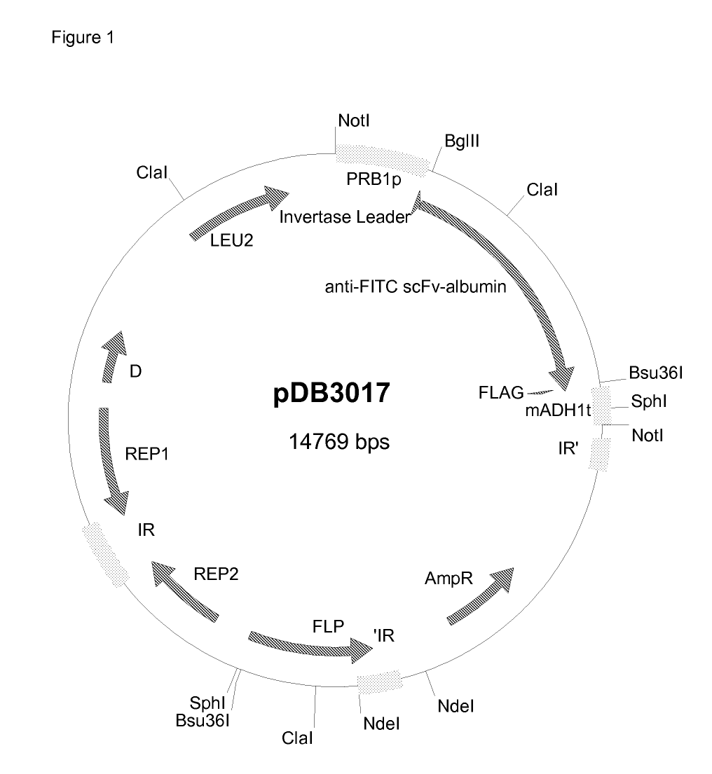

The sub-cloning plasmid pDB3006 was digested with the restriction endonuclease enzyme NotI and the 3.732 expression cassette was ligated into pSAC35 that had been digested with the restriction endonuclease enzyme NotI and using calf Intestinal phosphatase to produce the 16.303 kb plasmid pDB3017 (FIG. 1) that had the FITC (vHvL)-rHA expression cassette in the same orientation to the LEU2 gene.

The host strain used was DXY1, disclosed in S. M. Kerry-Williams et al. (1998) Yeast 14:161-169. DXY1 was transformed to leucine prototrophy with the FITC (vHvL)-rHA expression plasmid pDB3017. Yeast were transformed using a modified lithium acetate method (Sigma yeast transformation kit, YEAST-1, protocol 2; Ito et al, (1983), J. Bacteriol., 153, 16; Elble, (1992), Biotechniques, 13, 18). Transformants were selected on BMMD-agar plates, and subsequently patched out on BMMD-agar plates. The composition of BMMD is described by Sleep et al., (2002), Yeast, 18, 403. Cryopreserved stocks were prepared in 20% (w/v) trehalose from 10 mL BMMD shake flask cultures (24 hrs, 30° C., 200 rpm).

### Example 2

### Glycomapping

Identification of sites subjected to O-linked glycosylation was performed using the method below.

Microtubes (Bioquote Limited, York, UK) were set up containing 80 μL of 6 M Guanidine HCl Ul-tragrade (Sigma-Aldrich Company Ltd, Dorsett, UK), in 50 mM Tris-HCl (Sigma-Aldrich Company Ltd, Dorsett, UK) buffer pH8.0+2 mM EDTA (Sigma-Aldrich Company Ltd, Dorsett, UK), and 20 μL of FITC-rHA (Example 1) sample (˜30 mg/mL) were added to the tube. Samples were then reduced by adding 5 μL of 100 mM DTT 99% (Sigma-Aldrich Company Ltd, Dorsett, UK) (approx. 5 mM, actually 4.76 mM final concentration) to each tube, the samples were mixed and placed in an incubator for 1 ¼ hours in the dark at 37° C.

Samples were then alkylated to block free cysteines by adding 10 μL of 100 mM iodoacetamide Ultragrade (Sigma-Aldrich Company Ltd, Dorsett, UK) (approx. 10 mM, actually 8.69 mM final concentration) to each tube. The alkylation mixture was then mixed and incubated for 1 ¼ hours in the dark at 37° C.

After reduction and alkylation, 100 μL of 50 mM Tris-HCl (Sigma-Aldrich Company Ltd, Dorsett, UK) buffer pH8.0 and 400 μL of Laboratory Grade water were added to the reaction mix to ensure the final concentration of Guanidine in the digest was 1 M.

Modified sequencing grade Trypsin (Promega, Southampton, UK) for digestion was prepared as per the manufacturer's instructions to 1 mg/mL. Briefly 22 □L of supplied resuspension buffer was added to each 20 μg vial of Trypsin which was required. After re-suspension the Trypsin solutions were pooled prior to digestion. 10 μL of the Trypsin solution was added to each sample tube, the digestion mix was then mixed and pulse centrifuged. The reaction mixtures were then placed in a shaking incubator at 37° C. for 24 hours. After digestion was completed samples were frozen.

### ConA Extraction of Tryptic Glycopeptides

3 mL of ConA-Sepharose slurry (GE Healthcare, Bucks, UK) (mixed by inversion) was packed under gravity into 2 mL polystyrene chromatography columns (Pierce, Loughborough, UK) to yield approximately 1.5 mL bed volume. The columns were washed with 8 or more bed volumes of equilibration buffer, 100 mM NaAc, 100 mM NaCl, 1 mM MgCl₂, 1 mM MnCl₂, 1 mM_CaCl₂ pH5.5 (All Fisher Scientific Analytical Grade, Loughborough, UK). The tryptic digests were diluted with 5 mL of equilibration buffer and passed through the columns 3 times. The columns were then washed with 10 or more bed volumes of equilibration buffer. Retained ConA binding peptides were eluted with 2×4 mL of elution buffer, 100 mM NaAc, 100 mM NaCl, 0.5 M Methyl-α-D-Mannopyranoside, pH 5.5 (All Fisher Scientific Analytical Grade, Loughborough, UK). Eluate was then cleaned up by solid phase extraction (SPE) Briefly, one SPE column, containing 25 mg/mL 1000 A Styrene-Divinylbenzene (SDVB) resin (Biotage, Hertford, UK) was used per samples. Each column was wetted with 70% (v/v) Acetonitrile, 0.1% (v/v) Trifluoroacetic acid TFA (Riedel-deHaën, Loughborough, UK). Columns were then equilibrated with 0.1% (v/v) TFA (Riedel-deHaën, Loughborough, UK). The 8 mL of ConA eluent was then loaded onto the columns and allowed to slowly pass through at no more than 1 mL/min. Once all the samples had passed through the columns they were washed with 0.1% (v/v) Formic acid (Riedel-deHaen, Loughborough, UK). Bound peptides were then eluted with 0.5 mL 70% (v/v) Acetonitrile, 0.1% (v/v) Formic acid (Riedel-deHaen, Loughborough, UK) and collected in low bind microtubes.

After SPE clean up samples were dried down and stored at −20° C. prior to mass spectrometric analysis.

nanoHPLC msms Analysis

Glycopeptides were identified by nanoESI-HPLC-MS/MS. The dried digest mixtures were re-suspended in 40 □L 0.1% (v/v) Formic acid 98-100% (Merck Chemicals limited, Nottingham, UK) and 5 □L of the peptide mixture was separated on a 75 □M C18 100 Å PepMap™ column (Dionex UK Ltd, Camberley, UK), using an UltiMate nanoHPLC system with Famos auto-sampler (Dionex UK Ltd) at a flow rate of 300 nL/min. A 60 min gradient was used from 5% (v/v) B to 60% (v/v) B. (Buffer A 0.1% (v/v) Formic acid, Buffer B 0.1% (v/v) Formic acid and 70% (v/v) Acetonitrile (Rathburn Chemicals, Walkerbum, UK.).

ESI-MS/MS data were acquired on a QSTAR XL mass spectrometer (AppliedBiosytems, Warrington, UK) using the Analyst QSTM 1.1 software package in data dependent acquisition mode with automatic precursor ion selection of doubly and triply-charged ions. The MS survey scan was acquired at a mass range of 470-1800 m/z and MSMS spectra were acquired at a mass range of 100-1600 m/z. A maximum of three parent ions could be chosen for ms/ms at any time. An ion could be selected twice before being excluded for 60 seconds.

The acquired mass spectra were processed by Analyst QS™ 1.1 software package using the provided Mascot script. The mascot generic mass lists were then submitted to an in-house MASCOT (Matrix Science, London, UK) server for MS/MS ion database searching. The data were searched against a user created database containing several Novozymes' expressed proteins. The main search parameters that were used were: ±1.2 Da peptide ion mass tolerance and 0.6 Da fragment ion mass tolerance; masses were monoisotopic; proteolysis by trypsin; two missed cleavages were permissible; carbamidomethylation of cysteines was searched as a fixed modification; and variable modifications were: N-terminus of peptides changed from Gln to pyroGlu; oxidation of Met; acetylation of N-termini of proteins; and the mannosylation of serine, threonine and tyrosine, 1-4 mannoses were permitted per site. Any peptide matching the anti-FITC albumin fusion protein and containing at least one mannose modification was manually validated to confirm both the peptide and site of mannosylation were corrected.

### Results

The glycosite mapping experiment produced a number of mannosylated peptides from the anti-FITC scFv albumin fusion. One site of mannosylation (S190) has been confirmed down to the amino acid position.

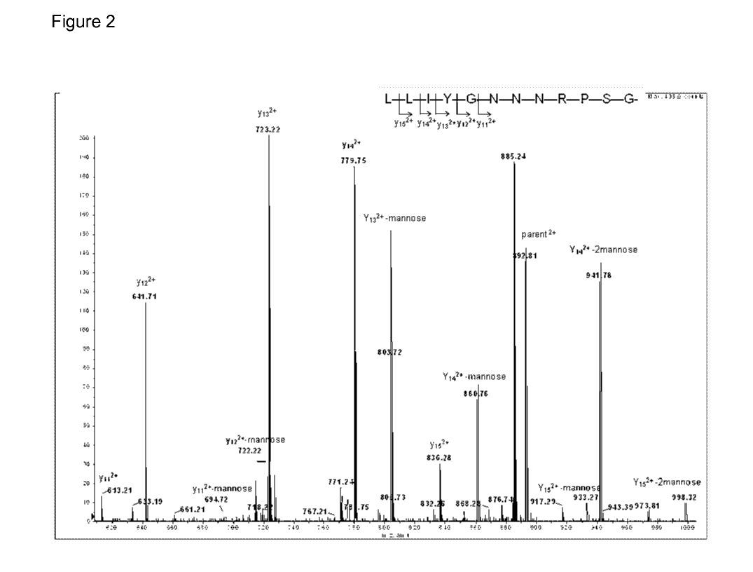

Tryptic peptides were ConA enriched before LCMSMS analysis. The majority of ions seen in the spectra corresponded to the un-mannosylated species (where the labile mannose had fallen off). However the presence of y11 and y12 with mannose still present confirms that at least one mannose is present at serine 190. The spectra is shown in FIG. 2.

### Example 3

### Fermentation

Fed-batch fermentations were carried out in a 10 L Sartorius Biostat C fermenter at 30° C.; pH was monitored and adjusted by the addition of ammonia or sulphuric acid as appropriate. The ammonia also provided the nitrogen source for the culture. The level of dissolved oxygen was monitored and linked to the stirrer speed, to maintain the level at >20% of saturation. Inocula were grown in shake flasks in buffered minimal media. For the batch-phase the culture was inoculated oculated into fermenter media (approximately 50% of the fermenter volume) containing 2% (w/v) sucrose. The feed stage was automatically triggered by a sharp rise in the level of dissolved oxygen. Sucrose was kept at growth-limiting concentrations by controlling the rate of feed to a set nominal growth rate. The feed consisted of fermentation media containing 50% (w/v) sucrose, all essentially as described by Collins. (Collins, S. H., (1990) Production of secreted proteins in yeast, in: T. J. R. Harris (Ed.) Protein production by biotechnology, Elsevier, London, pp. 61-77).

All fermentations were completed successfully and were good or perfect fermentations (Table 1).

TABLE 1
Anti FITC (vHvL)- rHA-Flag ® Fermentations..

| | | | | Batch | | | |
|---|---|---|---|---|---|---|---|
| Strain | | | | Phase | final | | Titre |
| [plasmid] | Batch No | CDW | Comment | (Hrs) | % s/n | Conductivity | mg/mL |
| DXY1 | 20083E008- | 104.3 | Good fermenta- | 51 | 57.5 | 8.02 | 5.08 |
| [3017] | 01 | | tion, | | | | |
| | | | feed rate capped | | | | |
| | | | at 2.7 ml/min | | | | |

### Example 4

### Purification

### Gel Permeation High Pressure Liquid Chromatography (GP-HPLC)

Protein concentrations were determined by GP-HPLC using a LC2010 HPLC system (Shimadzu) equipped with UV detection under Shimadzu VP7.3 client server software control. Injections of 25 μL were made onto a 7.8 mm id×300 mm length TSK G3000SWXL column (Tosoh Bioscience), with a 6.0 mm id×40 mm length TSK SW guard column (Tosoh Bioscience). Samples were chromatographed in 25 mM sodium phosphate, 100 mM sodium sulphate, 0.05% (w/v) sodium azide, pH 7.0 at 1 mL.min⁻¹, with a run time of 20 minutes. Samples were quantified by UV detection at 280 nm, by peak area, relative to a recombinant human albumin standard of known concentration (10 mg/mL) and corrected for their relative extinction coefficients.

### Supernatant Clarification

Culture supernatant from high cell density fed batch fermentations of the S. cerevicea strains DXY1 expressing the anti FITC scFv (vHvL)-rHA-FLAG protein was harvested by standard centrifugation, using a Sorvall RC 3 C centrifuge (DuPont).

### Protein Purification, Diafiltration and Concentration Steps

A three step chromatography procedure was used to prepare material suitable for bioanalysis, as described herein.

The first step uses a column (bed volume approximately 400 mL, bed height 11 cm) packed with AlbuPure™ matrix (ProMetic). This was equilibrated with 50 mM sodium phosphate, pH 6.0 and loaded with neat culture supernatant, at approximately pH 5.5-6.5, to approximately 20 mg fusion/mL matrix. The column was washed with approximately 5 column volumes each of 50 mM sodium phosphate, pH 6.0, 50 mM sodium phosphate, pH 7.0 and 50 mM ammonium acetate, pH 8.0, respectively. Bound protein was eluted using approximately two column volumes of 50 mM ammonium acetate, 10 mM octanoate, pH 7.0. The flow rate for the whole step was 154 mL/min.

For the second step, the eluate from the first step was diluted approximately two fold with water to give a conductivity of 2.5±0.5 mS/cm after adjustment to pH 5.5±0.3 with acetic acid. This was loaded onto a DEAE-Sepharose Fast Flow (GE Healthcare) column (bed volume approximately 400 mL, bed height 11 cm), equilibrated with 80 mM sodium acetate, 5 mM octanoate, pH 5.5. Loading was approximately 30 mg fusion/mL matrix. The column was washed with approximately 5 column volumes of 80mM sodium acetate, 5mM octanoate, pH 5.5. Followed by approximately 10 column volumes of 15.7 mM potassium tetraborate, pH 9.2. The bound protein was eluted using two column volumes of 110 mM potassium tetraborate, 200 mM sodium chloride, approximately pH 9.0. The flow rate was 183 mL/min during the load and wash steps, and 169 mL/min during the elution.

The eluate was concentrated and diafiltered against 20 mM Tris-HCL, 500 mM sodium chloride, pH 7.4, using a Pall Centramate Omega 10,000 NMWCO membrane, to give a final protein concentration of approximately 100 mg/mL.

For the third step, the concentrated and diafiltered eluate from the second step was adjusted with the addition of magnesium, calcium and manganese ions, to a final concentration of 1 mM, respectively. This was loaded onto a Con A Sepharose 4 B (GE Healthcare) column (bed volume approximately 160 mL, bed height 30 cm) at approximately 10 mg/mL matrix, equilibrated with 20 mM Tris-HCL, 500 mM sodium chloride, 1 mM magnesium chloride, 1 mM manganese chloride, 1 mM calcium chloride, pH 7.4. Unbound protein was eluted with 20 mM Tris-HCL, 500 mM sodium chloride, 1 mM magnesium chloride, 1 mM manganese chloride, 1 mM calcium chloride, pH 7.4. Bound protein was eluted with 20 mM Tris-HCL, 500 mM sodium chloride, 500 mM methyl manno-pyranoside, 1 mM magnesium chloride, 1 mM manganese chloride, 1 mM calcium chloride, pH 7.4. The flow rate during the load and elutions was 7.2 mL/min.

Samples were, when necessary, concentrated and diafiltered against 20 mM Tris-HCL, 500 mM sodium chloride, pH 7.4, using a Pall Centramate Omega 10,000 NMWCO membrane, to give a final protein concentration of approximately 10 mg/mL.

The table below summarizes representative %recovery of both the bound and unbound anti FITC scFv (vHvL)-rHA-FLAG protein, from the third step in the purification protocol, using the Con A Sepharose 4 B matrix.

| Strain | Unbound % Recovery | Bound % Purity |
|---|---|---|
| DXY1 | 78 | 5.2 |

### Example 5

### Disruption of Pmt Genes in S. cerevisiae

Creation of DXY1 trp1□

The host strain used to create a trp1□ strain was DXY1, as disclosed in S. M. Kerry-Williams et al. (1998) Yeast 14:161-169. The disruption that was created ensured the total removal of the native TRP1 sequence from the genome. This trp1□ strain was then used to facilitate the disruption of PMT1 and PMT4.

The synthetic DNA fragment to create a TRP1 disruption could be chemically synthesized with the DNA sequence provided in SEQ ID No.8. This chemically synthesised DNA could be digested with appropriate restriction endonuclease enzymes and ligated into an appropriate pUC19 based vector (Yanisch-Perron, et al. (1985) Gene. 33: 103-119).

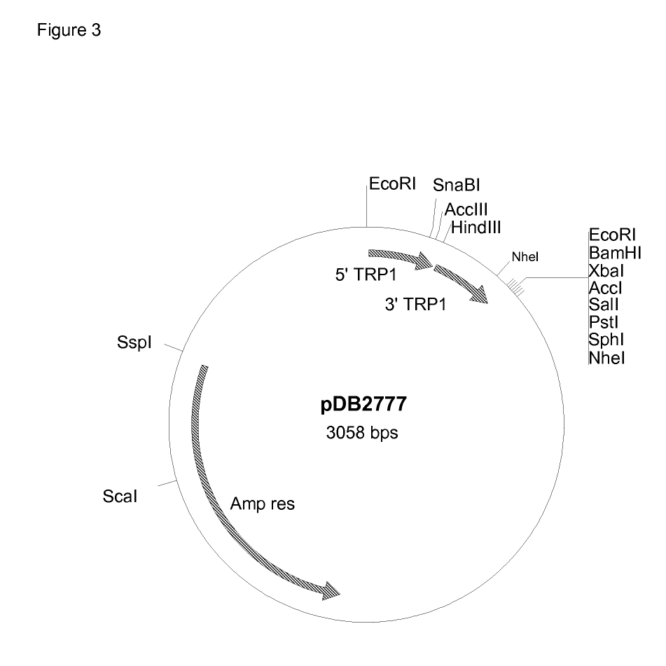

Plasmid pDB2777 (FIG. 3) was a pUC19 base vector that contained a piece of DNA identical to that described in SEQ ID No.8, which was liberated from its vector backbone with the restriction endonuclease enzyme EcoRI to release the TRPI disrupting fragment. This fragment along with the vector backbone as carrier DNA was used to transform DXY1 to tryptophan auxotrophy using a modified lithium acetate method (Sigma yeast transformation kit, YEAST-1, protocol 2; Ito et al. (1983) J. Bacteriol., 153, 16; Elble, (1992) Biotechniques, 13, 18). Yeast cells from the transformation were plated onto counter selective 0.3 g/l 5-fluoroanthranilic acid bactoagar plates (Toyn, et al. (2000) Yeast. 16:553-560). A tryptophan auxotroph was selected and the confirmation of the trp1□ strain genotype was confirmed by Southern blot analysis. This auxotroph as designated DXY1 trp1□.

Creation of the PMT1 Disrupted DXY1 strains DYP1 and DYP1 trp1□

The synthetic DNA fragment to create this disruption could be chemically synthesized with the DNA sequence provided in SEQ ID No.9. This chemically synthesized DNA would contain the 5′ of the PMT1 gene from the NcoI restriction endonuclease enzyme site at bp 130 to a natural HindIII restriction endonuclease enzyme site within PMT1 gene at bp 911, and then from the natural HindIII site at bp 1595 to the second NcoI restriction endonuclease enzyme site at bp 2136. This chemically synthesized DNA could be digested with appropriate restriction endonuclease enzymes and ligated into an appropriate pBR322 based vector (Bolivar, et al. (1977) Gene, 2, 95-113).

The TRP1 auxotrophic selective marker could be chemically synthesized with the DNA sequence provided in SEQ ID No.10. This chemically synthesized DNA would contain the HindIII restriction endonuclease enzyme sites at either end of the TRP1 auxotrophic selective marker to facilitate ease of cloning.

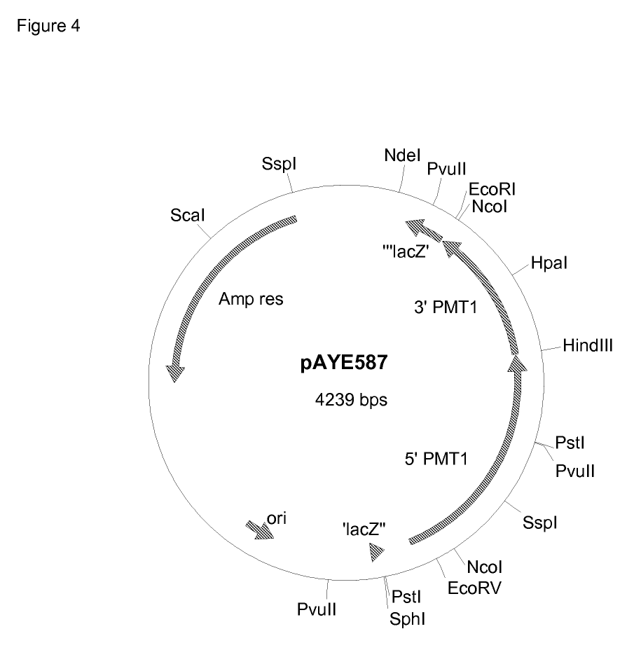

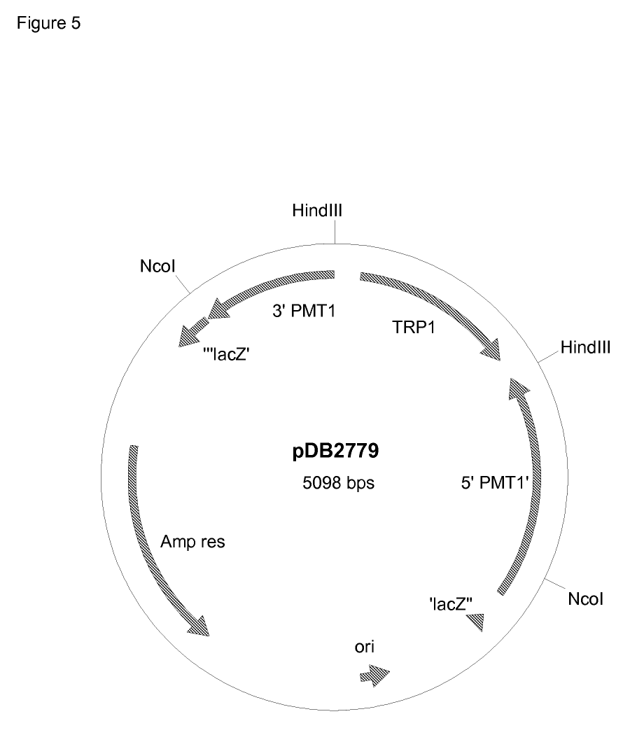

Plasmid pAYE587 (FIG. 4) was a pBR322 based vector that contained a piece of DNA identical to that described in SEQ ID No.9. This plasmid was linearised with the restriction endonuclease enzyme HindIII and treated with calf intestinal phosphatase to produce a vector into which was ligated a piece of DNA identical to that described in SEQ ID No.10, containing the TRP1 auxotrophic selective marker, that had also been digested with the restriction endonuclease enzyme HindIII. In plasmid pDB2779 (FIG. 5) the TRP1 auxotrophic selective marker was orientated in the opposite direction to the PMT1 ORF. The plasmid pDB2779 was then digested with the restriction endonuclease enzyme NcoI to release the 2.181 kb pmt1::TRP1 disruption fragment.

The pDB2779 2.181 kb pmt1::TRP1 disruption fragment was used along with the pDB2779 backbone, which acted as carrier DNA, to transform the yeast strain DXY1 trp1□to tryptophan prototrophy using a modified lithium acetate method (Sigma yeast transformation kit, YEAST-1, protocol 2; Ito et al. (1983) J. Bacteriol., 153, 16; Elble, (1992) Biotechniques, 13, 18). A tryptophan prototroph was selected and the confirmation of the pmt1::TRP1 strain genotype was confirmed by Southern blot analysis. This prototroph was designated DYP1.

To be able to disrupt multiple PMT genes DYP1 was also made DYP1 pmt1::trp1□. This was achieved by removing the TRP1 auxotrophic selective marker from the middle of the disrupted pmt1 gene by using the 1.322 kb NcoI fragment containing the 5′ and 3′ ends of PMT1 from plasmid pAYE587.

This 1.322 kb NcoI fragment from pAYE587, along with the vector backbone as carrier DNA was used to transform DYP1 to tryptophan auxotrophy using a modified lithium acetate method (Sigma yeast transformation kit, YEAST-1, protocol 2; Ito et al. (1983) J. Bacteriol., 153, 16; Elble, (1992) Biotechniques, 13, 18). Yeast cells from the transformation were plated onto counter selective 0.3 g/l 5-fluoroanthranilic acid bactoagar plates (Toyn, et al. (2000) Yeast. 16:553-560). A tryptophan auxotroph was selected and the confirmation of the trp1□ strain genotype was confirmed by Southern blot analysis. This auxotroph was designated DYP1 trp1\tilde{□}

### Creation of the PMT4 Disrupted DXY1 Strain DYP4

The synthetic DNA fragment to create a PMT4 disruption could be chemically synthesized with the DNA sequence provided in SEQ ID No.11. This chemically synthesized DNA would contain the 5′ of the PMT4 gene from the BglII restriction endonuclease enzyme site at by 211 to an analogous site within the sequence comparable to PMT1, into which an engineered HindIII site was created, and then from a second region analogous to the second HindIII in PMT1 a second HindIII site was created in the 3′ region of PMT4 to the XbaI restriction endonuclease enzyme site at by 2250. This chemically synthesized DNA could be digested with appropriate restriction endonuclease enzymes and ligated into an appropriate pMCS5 based vector (Hoheisel, J.(1994) BioTechniques 17(3) 456-459).

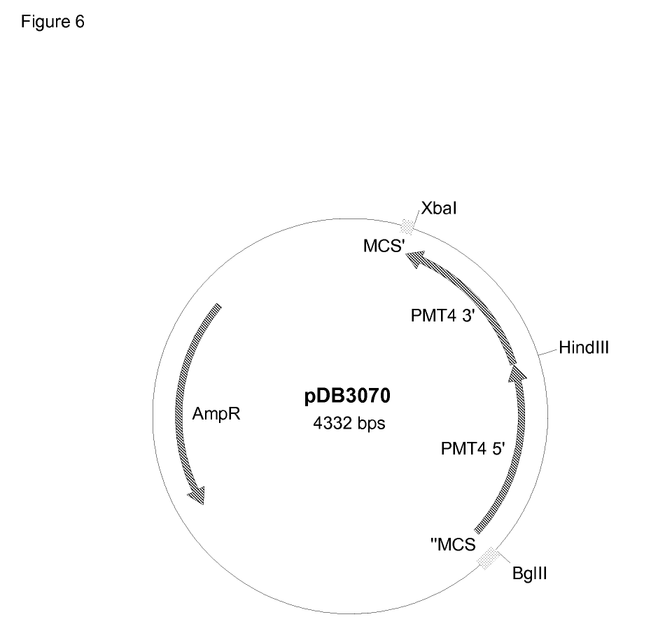

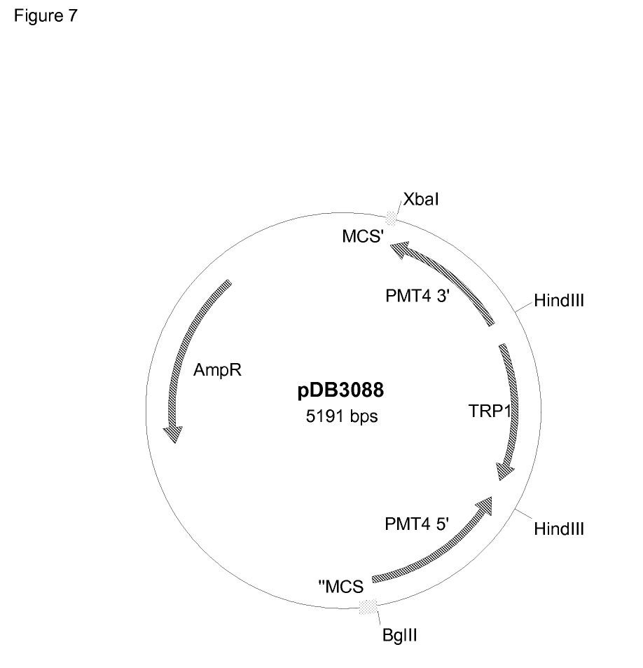

Plasmid pDB3070 (FIG. 6) was a pMCS5 based vector that contained a piece of DNA identical to that described in SEQ ID No.11. This plasmid was linearised with the restriction endonuclease enzyme HindIII, and treated with calf intestinal phosphatase to produce a vector into which was ligated a piece of DNA identical to that described in SEQ ID No.10, containing the TRP1 auxotrophic selective marker, that had also been digested with the restriction endonuclease enzyme HindIII. This produced the plasmid pDB3088 (FIG. 7) where the TRP1 auxotrophic selective marker was orientated in the opposite direction to the PMT4 ORF. The plasmid pDB3088 was then digested with the restriction endonuclease enzymes BglII and XbaI to release the 2.231 kb pmt4::TRP1 disruption fragment.

The pDB3088 2.231 kb pmt4::TRP1 disruption fragment was used along with the pDB3088 backbone, which acted as carrier DNA, to transform the yeast strain DXY1 trp1□to tryptophan prototrophy using a modified lithium acetate method (Sigma yeast transformation kit, YEAST-1, protocol 2; Ito et al. (1983) J. Bacteriol., 153, 16; Elble, (1992) Biotechniques, 13, 18). A tryptophan prototroph was selected and the confirmation of the pmt4::TRP1 strain genotype was confirmed by Southern blot analysis. This prototroph was designated DYP4.

To be able to disrupt multiple PMT genes DYP4 was also made DYP4 pmt4::trp1□. This was achieved by removing the TRP1 auxotrophic selective marker from the middle of the disrupted pmt4 gene by using the 1.372 kb BglII/XbaI fragment containing the 5′ and 3′ ends of PMT4 from plasmid pDB3070.

This 11.372 kb BglII/XbaI fragment from pDB3070 along with the vector backbone as carrier DNA was used to transform DYP4 to tryptophan auxotrophy using a modified lithium acetate method (Sigma yeast transformation kit, YEAST-1, protocol 2; Ito et al. (1983) J. Bacteriol., 153, 16; Elble, (1992) Biotechniques, 13, 18). Yeast cells from the transformation were plated onto counter selective 0.3 g/l 5-fluoroanthranilic acid bactoagar plates (Toyn, et al. (2000) Yeast. 16:553-560). A tryptophan auxotroph was selected and the confirmation of the trp1□ strain genotype was confirmed by Southern blot analysis. This auxotroph was designated DYP4trp1\tilde{□}.

### Example 6

### The Effect Selective Mutations on Glycosylation

### Construction of the S190A FITC-rHSA Fusion

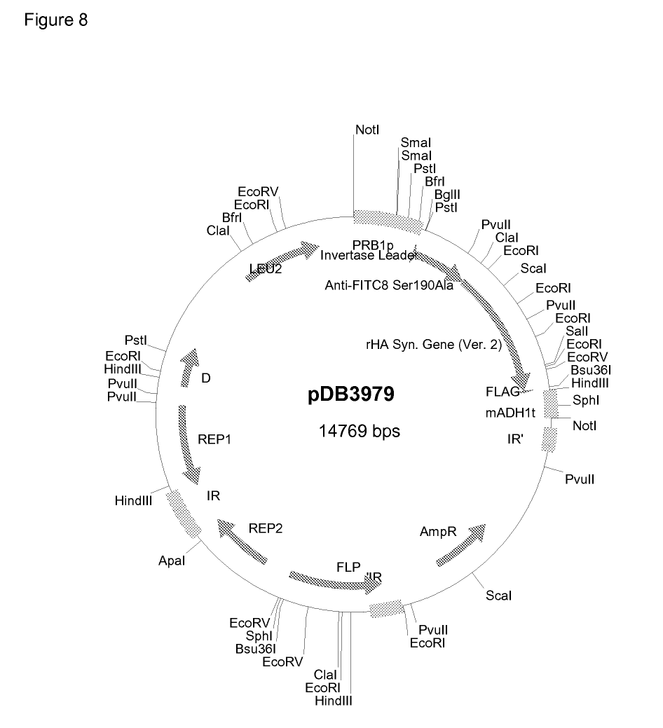

Briefly, the point mutant S190A was constructed as was the parental FITC fusion, described in detail in example 1. The only difference being the sequence of the scFv. The fermentation and purification methodologies were as described in examples 3 and 4, respectively. FIG. 8 shows the expression plasmid in detail. Expressing this expression plasmind as disclosed above provided the S190A FITC scFv-rHSA fusion where the S190A FITC scFv has the sequence SEQ ID NO: 12.

The fermentation and purification methodologies were as described in examples 3 and 4, respectively.

The ConA binding assay was as follows; Con A Sepharose (GE Healthcare, Bucks,UK) affinity chromatography was used to isolate mannosylated proteins from recombinant scFv albumin fusions: 3% (w/v) (approx.) scFv albumin fusions were diluted 1:1 with Con A dilution buffer (200 mM sodium acetate, 85 mM sodium chloride, 2 mM magnesium chloride, 2 mM manganese chloride, 2 mM calcium chloride ph5.5 (All Fisher Scientific Analytical Grade, Loughborough, UK), and 350 microL (˜5 mg) loaded onto an equilibrated 2 mL Con A Sepharose column which was then washed (5×4 mL) with Con A equilibration buffer (100 mM sodium acetate, 100 mM sodium chloride, 1 mM magnesium chloride, 1 mM manganese chloride, 1 mM calcium chloride ph5.5 (All Fisher Scientific Analytical Grade, Loughborough, UK). The column was eluted with 6 mL Con A elution buffer (100 mM sodium acetate, 100 mM sodium chloride, 0.5 M methyl-α-D-mannopyranoside ph5.5 (All Fisher Scientific Analytical Grade, Loughborough, UK). Triplicate columns were run for each sample.

Con A loads and eluates were quantified by Bradford assay using a rHA standard curve and the Con A binding material recovered in the eluate expressed as a percentage of the load.

The rHA standard used was Recombumin® available from Novozymes Biopharma UK. The ConA binding assay data shows that the selective substitution of the identified site of glycosylation leads to a significant reduction in the level of glycosylation. There is also a slight advantage to the use of either a pmt1 and/or a pmt4 disrupted host strain to further enhance the observed reduction in glycosylation.

TABLE 2

| | | % w/w bound |
|---|---|---|
| Molecule | Strain | to Con A column |
| scFv(vHvL)- | DXY1 | 6.9 |
| rHA | | |
| scFv(vHvL)- | DXY1 | 3.6 |
| rHA S190A | | |
| Mutation | | |
| scFv(vHvL)- | DYP1 | 3.1 |
| rHA S190A | | |
| Mutation | | |
| scFv(vHvL)- | DYP4 | 3.4 |
| rHA S190A | | |
| Mutation | | |

### Example 7

### Identification of Additional Sites of Glycosylation in an Immunoglobulin

Using the methodologies described in detail in Example 2 further novel sites of glycosylation have been identified down to the amino acid position from muteins purified from the three described host strains described in Example 5.

In addition to the mass spectrometric analysis (described in Example 2) Beta Elimination/Michael Addition (BEMAd) was done

### Beta Elimination Michael Edition

Two methods of (BEMAd) were employed to obtain glycosite information. BEMAd 1 (Rademaker, et al Anal Biochem 1998, Mar 15, 257(2) p149) employed ammonium hydroxide as the active ingredient and BEMAd3 (Zheng et al, et al Talanta 78 (2009) 358-363) employed dimethylamine.

### BEMAd1

Lyophilised digested tryptic peptides dried down after digestion were resuspended in 446.4 μL 28% NH₄OH (Sigma-Aldrich Company Ltd, Dorsett, UK) and 53.6 μL, the equivalent of 500 μL 25% NH₄OH. Samples were then incubated at 45° C. for 16 hrs. After incubation samples were dried down ready for ZipTip clean up.

### BEMAd3

Lyophilised digested tryptic peptides were resuspended in 195 μL of 40% dimethylamine (Sigma-Aldrich Company Ltd, Dorsett, UK). Samples were then incubated at 55° C. for 6 hours before being lyophilised prior to ZipTip clean up.

### ZipTip Sample Clean Up

ZipTip_{C18}P10 (Millipore (U.K.) Limited, Watford, UK) were used to desalt the lyophilised BEMAd samples. All liquid handling for ZipTip desalting was carried out using a P20 Gilson pipette (Scientific Laboratory Supplies Limited, Nottingham, UK). Lyopholised BEMAd samples were resuspended in 20 μL 0.1% formic acid 98-100% (Merck Chemicals limited, Nottingham, UK) directly before ZipTip clean-up. ZipTips were then wetted in 200 μL 0.1% Formic acid and 70% Acetonitrile (Rathburn Chemicals, Walkerburn, UK.) before being equilibrated in 200 μL 0.1% Formic acid. After equilibration resuspended samples were aspirated and fully expelled from ZipTips at least 10 times to ensure full sample binding to C18 matrix. ZipTips were then washed in at least 100 μL 0.1% formic acid. After washing samples were eluted using aspirating and fully expelling 20 μL 0.1% Formic acid and 70% Acetonitrile a minimum of 10 times. Eluted peptides were then dried down and stored at −20° C. until required for HPLC MSMS analysis.

nanoHPLC msms Analysis

BEMAd labelled samples were separated on an 80 min gradient from 5% buffer B to 80% buffer B, but otherwise conditions were as described in Example 2.

Similarly, the acquired mass spectra were processed by Analyst QS™ 1.1 software package using the provided Mascot script. The mascot generic mass lists were then submitted to an inhouse MASCOT (Matrix Science, London, UK) server for MS/MS ion database searching. The data were searched against a user created database containing several Novozymes' expressed proteins. The main search parameters that were used were: ±1.2 Da peptide ion mass tolerance and 0.6 Da fragment ion mass tolerance; masses were monoisotopic; proteolysis by trypsin; two missed cleavages were permissible; carbamidomethylation of cysteines was searched as a fixed modification; and variable modifications were dependent on whether samples were BEMAd treated and which BEMAd method was used. Non BEMAd labelled samples: N-terminus of peptides changed from Gln to pyroGlu; oxidation of Met; acetylation of N-termini of proteins; and the mannosylation of serine, threonine and tyrosine, 1-4 mannoses were permitted per site; BEMAd1: Acetyl (Protein N-term),Deamidated (NQ),Gln->pyro-Glu (N-term Q),Oxidation (M),Ser Dehydro (S),Thr Dehydro (T),Dehydrated (S),Dehydrated (T); BEMAd2: Acetyl (Protein N-term),Deamidated (NQ),Gln->pyro-Glu (N-term Q),Oxidation (M),Ser Dehydro (S),Thr Dehydro (T),BEMAD 3 (ST). Any peptide matching the anti-FITC albumin fusion protein and containing at least one mannose modification was manually validated to confirm both the peptide and site of mannosylation were corrected.

1. A method for preparing a polypeptide comprising an antibody sequence, said polypeptide having a low degree of O-linked glycosylation, comprising the step of:
a. providing a nucleic acid sequence encoding a polypeptide comprising an antibody sequence;
b. modifying the nucleic acid sequence so that at least one amino acid residue selected among S, T and Y and subjected to O-linked glycosylation is substituted or deleted;
c. introducing the modified nucleic acid sequence in a suitable host cell so that the modified nucleic acid sequence is capable of being expressed in the host cell;
d. growing the host cell under conditions leading to expression of the polypeptide encoded by the modified nucleic acid sequence, and
e. recovering the polypeptide.

2. The method of claim 1, wherein the antibody sequence comprises framework sequences.

3. The method of claim 1, wherein the polypeptide is selected among antibodies, fragments or variants thereof, or fusion proteins comprising an antibody, a fragment or variant thereof.

4. The method according to claim 1, wherein the at least one amino acid residue subjected to O-linked glycosylation is selected among amino acids having the following position according to the Kabat numbering: L56 and amino acids in positions corresponding to positions 7, 72, 191 or 206 in SEQ ID NO: 12.

5. The method of claim 4, wherein the amino acid numbered L56 according to the Kabat numbering is substituted with another amino acid selected among: G, A, V.

6. The method according to claim 1, wherein the host cell is a fungal host cell.

7. The method of claim 6, wherein the host cell is selected among: Aspergillus sp., such as A. nidulans, A. niger, A. awamori and A. oryzae; Trichoderma sp., such as T. reeseii, T. Longibrachiatum and T. virdee; Penicillum sp. such as P. notatum and P. chrysogenum; Fusidium sp., Fusarium sp., Scizophyllum sp. Mucor sp, Rhizopus sp., Saccharomyces sp. such as S. cerevisiae and S. ovarum, Zygosaccharomyces sp., Schizosaccharomyces sp. such as S. pombe, Klyveromyces sp. such as K. lactis, Candida sp. such as C. albicans, Pichia sp. and Hansenula sp.

8. The method of claim 7, wherein the host cell is selected among Saccharomyces serevisiae, Schizosaccharomyces pombe, Klyveromyces lactis, Pichia pastoris, Aspergillus nidulans, Aspergillus niger, Aspergillus oryzae and Trichoderma reseii.

9. A polypeptide obtainable according to the method of claim 1.

10. A composition comprising a polypeptide prepared according to the method of claim 1.

11. The composition of claim 10, wherein the polypeptide is an antibody, a fragment or variant thereof, or a fusion protein comprising an antibody, a fragment or variant thereof.
